(19) United States
(12) Patent Application Publication
(10) Pub. No.: US 2025/0091976 A1
(43) Pub. Date: Mar. 20, 2025

(54) METHOD OF PRODUCING FORMALDEHYDE

(71) Applicant: JOHNSON MATTHEY DAVY TECHNOLOGIES LIMITED, London (GB)

(72) Inventor: Paul John CASSIDY, London (GB)

(21) Appl. No.: 18/725,990

(22) PCT Filed: Mar. 1, 2023
(86) PCT No.: PCT/GB2023/050463

§ 371 Date: Jul. 1, 2024

(30) Foreign Application Priority Data
Mar. 21, 2022 (GB) 2203921.8

(51) Int. Cl. C07C 45/00; C01B 3/22; C07C 29/151

(52) U.S. Cl. CPC C07C 45/002; C01B 3/22; C07C 29/1518; C01B 2203/0277; C01B 2203/1041; C01B 2203/1223

(57) ABSTRACT

A method of producing formaldehyde, the method comprising: generating electrolytic hydrogen from the electrolysis of water; providing a feedstock gas stream comprising the electrolytic hydrogen and one or both of carbon monoxide and carbon dioxide; converting at least a portion of the feedstock gas to methanol; converting at least a portion of the methanol to formaldehyde and hydrogen; separately recovering at least some of the formaldehyde and at least some of the hydrogen; and recycling at least some of the recovered hydrogen to the feedstock gas stream.

### FIELD OF THE INVENTION

The invention relates to a method of producing formaldehyde.

### BACKGROUND OF THE INVENTION

The traditional formaldehyde production process involves the oxidation of methanol to produce formaldehyde and water, i.e.

2CH₃OH+O₂→2CH₂O+2H₂O

The typical catalyst employed comprises a mixture of iron oxide with either molybdenum oxide or vanadium oxide, and the reaction is typically carried out at 250-400° C. with a high methanol conversion rate of 98-99%. Silver-based catalysts can also be employed. Two chemical reactions on the silver-based catalyst simultaneously produce formaldehyde: that shown above and the dehydrogenation reaction:

CH₃OH→CH₂O+H₂

However, silver-based catalysts usually operate at a higher temperature, about 650° C., and exhibit a lower methanol conversion rate than iron oxide-based catalysts.

U.S. Pat. No. 6,472,566 discloses apparatus for preparing formaldehyde from methanol by dehydrogenation in a reactor in the presence of a catalyst at a dehydrogenation temperature in the range from 300 to 1000° C., employing a carrier gas stream at a temperature above the dehydrogenation temperature.

Formaldehyde production currently accounts for approximately 30% of current worldwide methanol production and is the leading consumer of methanol. Methanol can be produced over copper-based catalysts from syngas, a fuel gas mixture containing, inter alia, carbon monoxide, carbon dioxide and hydrogen. Today, the most widely used catalyst is a mixture of copper and zinc oxides, supported on alumina, as first used by ICI in 1966. At 5-10 MPa and 250° C., the reaction is characterized by high selectivity (>99.8%). The reactions to form methanol may be depicted as follows:

CO+2H₂→CH₃OH

CO₂+3H₂→CH₃OH+H₂O

The current “green route” to produce methanol is from hydrogen generated by electrolysis using renewable energy and waste carbon dioxide. The main drawback of this process is the high cost of hydrogen produced through electrolysis.

The present invention seeks to tackle at least some of the problems associated with the prior art or at least to provide a commercially acceptable alternative solution thereto.

### SUMMARY OF THE INVENTION

One aspect of the present disclosure is directed to a method of producing formaldehyde, the method comprising:
generating electrolytic hydrogen from the electrolysis of water;
providing a feedstock gas stream comprising the electrolytic hydrogen and one or both of carbon monoxide and carbon dioxide;
converting at least a portion of the feedstock gas to methanol;
converting at least a portion of the methanol to formaldehyde and hydrogen;
separately recovering at least some of the formaldehyde and at least some of the hydrogen; and
recycling at least some of the recovered hydrogen to the feedstock gas stream.

### BRIEF DESCRIPTION OF THE DRAWINGS

FIG. 1 shows a flow diagram of a plant suitable for carrying out the method of producing formaldehyde according to the present invention.

FIG. 2 shows a flow diagram of another plant suitable for carrying out the method of producing formaldehyde according to the present invention.

### DETAILED DESCRIPTION OF THE INVENTION

In a first aspect, the present disclosure is directed to a method of producing formaldehyde, the method comprising:
generating electrolytic hydrogen from the electrolysis of water;
providing a feedstock gas stream comprising the electrolytic hydrogen and one or both of carbon monoxide and carbon dioxide;
   converting at least a portion of the feedstock gas to methanol;
   converting at least a portion of the methanol to formaldehyde and hydrogen;
   separately recovering at least some of the formaldehyde and at least some of the hydrogen; and
   recycling at least some of the recovered hydrogen to the feedstock gas stream.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

Advantageously, the recycling of the recovered hydrogen may result in up to a third of the hydrogen consumed during the method being saved. When hydrogen in the feedstock gas stream is produced using electrolysis, this may result in a significant cost saving to the method. Surprisingly, such cost savings in the production of hydrogen may outweigh any disadvantages associated with generating formaldehyde via a dehydrogenation route, e.g. higher temperatures, lower conversion rates etc. Accordingly, in comparison to conventional formaldehyde production methods, the method of the present invention may be lower cost and/or more environmentally friendly.

The term “formaldehyde” as used herein may encompass an organic compound with the following molecular formula:

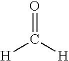

The systemic name for formaldehyde is methanal.

The method comprises providing a feedstock gas stream comprising hydrogen (i.e. molecular hydrogen, H₂) and one or both of carbon monoxide (i.e. CO) and carbon dioxide (i.e. CO₂).

The hydrogen in the feedstock gas stream is electrolytic hydrogen generated by the electrolysis of water. Any suitable electrolysis equipment may be used. For example, the electrolysis equipment may comprise an alkaline electrolyser, a polymer electrolyte membrane (PEM) electrolyser, a solid oxide electrolysis cell (SOEC) electrolyser, an alkaline exchange membrane (AEM) electrolyser or an anion exchange membrane electrolyser.

The feedstock gas stream may comprise species other than hydrogen, carbon monoxide and carbon dioxide, e.g. water, sulphur-containing species, tar and soot. Typically, the total amount of hydrogen, carbon monoxide and carbon dioxide makes up at least 90 vol. % of the feedstock gas stream at the point of being converted to methanol.

Prior to converting at least a portion of the feedstock gas to methanol, the feedstock gas stream may be purified to remove contaminants which might otherwise poison catalysts used for the methanol conversion and/or contaminate the methanol product. For example, the purification may comprise removing sulphur-containing species (e.g. H₂S) and/or chlorine-containing species (e.g. HCl) from the feedstock gas stream. The purification may also comprise removing solid species such as tar and soot from the feedstock gas stream, for example via a de-coking step.

Typically, the feedstock is pressurised. Pressurisation may increase the methanol conversion rate and/or yield.

The method comprises converting at least a portion of the feedstock gas to methanol. Preferably, at least 50 vol. % of the feedstock gas is converted to methanol, more preferably at least 75 vol. %, even more preferably at least 90 vol. %, still even more preferably at least 95 vol. %, still even more preferably substantially all of the feedstock gas is converted to methanol.

The reaction generates a product gas mixture containing methanol and steam, and unreacted feedstock. Unreacted feedstock may be separated from methanol by cooling the product gas mixture to condense the methanol and water, which may be recovered as a crude methanol product.

Prior to converting at least a portion of the methanol to formaldehyde and hydrogen, the crude methanol product is typically purified, for example via one or more steps of distillation. Such distillation may serve to purify the methanol to a purity greater than 90% vol., preferably greater than 95% vol., more preferably greater than 99% vol. prior to the formaldehyde conversion step.

The method comprises converting at least a portion of the methanol to formaldehyde and hydrogen, i.e. formaldehyde is produced via the dehydrogenation route. This route operates in the absence of added oxygen in a dehydrogenation reactor containing a dehydrogenation catalyst. If oxygen is present, some of the methanol may also be simultaneously converted to formaldehyde via the oxidation route. However, the oxidation route is preferably kept to a minimum. Preferably, at least 50 vol. % of the methanol is converted to formaldehyde and hydrogen, more preferably at least 75 vol. %, even more preferably at least 90 vol. %, still even more preferably at least 95 vol. %, still even more preferably substantially all of the methanol is converted to formaldehyde and hydrogen.

The method comprises separately recovering at least some of the formaldehyde and at least some of the hydrogen. By “separately recovering” it is meant that the species are separated from both a formaldehyde product stream recovered from the dehydrogenation reactor and each other. Suitable techniques for separately recovering the formaldehyde and hydrogen are known in the art. The formaldehyde may be recovered, for example, via absorption in an absorption unit. The absorption unit may extract the formaldehyde in the formaldehyde product stream into, for example, water to produce an aqueous formaldehyde solution (formalin), or a urea solution to produce a urea-formaldehyde concentrate (UFC). Absorbing formaldehyde into such solutions may prevent the spontaneous polymerisation to paraformaldehyde. The absorption may be performed using an absorption tower, which may contain a selection of packing, trays and other features to promote absorption, and cooling water may be used to provide the product at a temperature in the range 20-100° C. The absorption is typically carried out at a slightly lower pressure than the reactor. The recovered absorbed formaldehyde may be passed to a downstream process, for example a process to produce urea, or a process to manufacture one or more of urea formaldehyde resin, melamine resin, phenol formaldehyde resin, polyoxymethylene plastics, 1,4-butanediol, and methylene diphenyl diisocyanate.

The hydrogen may be recovered, e.g. from the formaldehyde absorption unit off-gas using a membrane separation unit and/or a pressure swing adsorption (PSA) unit. The formaldehyde may be recovered before the hydrogen, after the hydrogen or at the same time as the hydrogen.

The recovery of the formaldehyde and hydrogen from the formaldehyde product stream generates a waste stream. The waste stream may comprise, for example, unconverted methanol and/or unconverted feedstock gas and/or water. At least a portion of the unconverted methanol may be recovered and recycled for use in the step of converting at least a portion of the methanol to formaldehyde and hydrogen.

The method comprises recycling at least some of the recovered hydrogen to the feedstock gas stream, preferably the majority (i.e. at least 50 vol. %) of the recovered hydrogen, more preferably at least 75 vol. % of the recovered hydrogen, even more preferably at least 90 vol. % of the recovered hydrogen, still even more preferably at least 95 vo. % of the recovered hydrogen, still even more preferably substantially all of the recovered hydrogen. The recycling of the recovered hydrogen enables the recovered hydrogen to be used in the methanol conversion step.

Providing a feedstock gas stream comprises generating hydrogen from the electrolysis of water. The electrolysis of water is preferably carried out using renewable energy. Hydrogen generated by electrolysis, in particular where the electrolysis is powered by renewable energy, produces low levels of carbon dioxide, typically substantially no carbon dioxide. As a result, the method is more environmentally friendly in comparison to conventional formaldehyde production methods.

The feedstock gas stream preferably comprises one or more of: carbon dioxide recovered from a waste stream, carbon dioxide recovered from a flue gas, and carbon dioxide recovered by direct air capture. As a result, the method is more environmentally friendly in comparison to conventional production methods.

In some embodiments, the feedstock gas stream comprises, or consists of, electrolytic hydrogen and carbon dioxide from one or more of these sources, or another source of carbon dioxide that would otherwise be vented to atmosphere.

In some embodiments, the feedstock gas stream comprises, or consists of, electrolytic hydrogen and a syngas containing hydrogen, carbon dioxide and carbon monoxide generated by the conversion of hydrocarbons, coal, plastics, biomass or municipal waste in a syngas generation unit. These embodiments are particularly useful where the syngas produced by the syngas generation unit is hydrogen deficient, e.g. has a stoichiometry number less than 1.9, or less than 1.8, or less than 1.7. The syngas from the syngas generation unit may comprise sulphur-containing gas, e.g. hydrogen sulphide (i.e. H₂S). In that case, the method preferably further comprises removing sulphur-containing gas from the syngas and/or feedstock gas stream before converting a portion of the feedstock gas stream to methanol. The components of the syngas will vary depending on its method of manufacture and the starting materials used.

The method may include gasification of a carbonaceous material, such as coal, plastics, biomass or municipal waste, in the syngas generation unit to provide the syngas. Gasification of a carbonaceous material is particularly effective at providing syngas with the correct types and amounts of starting materials for methanol conversion. Gasification is a technique known in the art. During gasification, the carbonaceous material is treated with oxygen and steam while also being heated (and in some cases pressurized). Advantageously, heat may be recovered from the gasification for use in other steps of the method.

The carbonaceous material preferably comprises one or more of biomass, municipal waste, and plastics. Such carbonaceous materials may reduce the environmental burden on the process.

In preferred embodiments, providing a feedstock gas comprises combining electrolytic hydrogen and waste CO₂, or combining electrolytic hydrogen with a syngas. The electrolytic hydrogen is generated from the electrolysis of water, preferably using renewable electricity. The waste CO₂ is preferably recovered from combustion processes. The syngas is preferably generated from the gasification of a carbonaceous material, wherein the gasification uses oxygen generated from the electrolysis of the water. Such a method may be particularly environmentally friendly in comparison to conventional methods.

Converting at least a portion of the feedstock gas to methanol is preferably carried out using a feedstock gas with a stoichiometry number R of about 2, preferably in the range 1.95 to 2.05, wherein R is defined by the following formula:

R=([H₂]−[CO₂])/([CO₂]+[CO]).

This may provide a particularly high methanol conversion rate.

The feedstock gas may comprise both carbon monoxide and carbon dioxide, or may comprise carbon dioxide without any carbon monoxide.

Converting at least a portion of the hydrogen and at least a portion of the one or both of carbon monoxide and carbon dioxide to methanol comprises contacting the feedstock gas stream with a catalyst. The contacting preferably occurs at a temperature of from 200 to 330° C., preferably 225 to 315° C. and/or at a pressure of from 5 to 10 MPa. The catalyst preferably comprises alumina-supported copper and zinc oxides. The use of such a catalyst, in particular under such conditions, may be particularly suitable for producing methanol, and may result in a high conversion rate.

Prior to converting at least a portion of the methanol to formaldehyde and hydrogen, preferably the methanol is purified using distillation. Purification of the methanol prior to conversion may increase the efficiency of the reaction and/or reduce poisoning of any catalysts used for the conversion. Distillation is a particularly suitable technique for purifying methanol. Methanol may be recovered from the feedstock gas stream by the distillation.

Converting at least a portion of the methanol to formaldehyde and hydrogen preferably comprises contacting the methanol with a catalyst. In order to achieve an ecologically and economically useful industrial process for the dehydrogenation of methanol, the following prerequisites have to be met: the strongly endothermic reaction has to be carried out at high temperatures so as to be able to achieve high conversions; competing secondary reactions have to be suppressed in order to achieve satisfactory selectivity to formaldehyde (without catalysis, the selectivity for the formation of formaldehyde is less than 10% at conversions over 90%); and residence times have to be short and the cooling of the reaction products has to be rapid in order to lessen the decomposition of the formaldehyde which is not thermodynamically stable under the reaction conditions.

Various methods of carrying out this reaction have been proposed; and any of these may be used in the present method. For example, DE-A-37 19 055 describes a process for preparing formaldehyde from methanol by dehydrogenation in the presence of a catalyst at elevated temperature. The reaction is carried out in the presence of a catalyst comprising at least one sodium compound at a temperature of from 300° C. to 800° C.

The methanol may be provided to the dehydrogenation reactor in a carrier gas, such as nitrogen, at a temperature above the reaction temperature as described in U.S. Pat. No. 6,472,566.

The contacting preferably occurs at a temperature of from 300° C. to 800° C., more preferably 450 to 650° C., and/or at a pressure of up to 5 MPa. Such conditions may increase the conversion rate and/or increase the yield.

The catalyst for converting at least a portion of the methanol to formaldehyde and hydrogen may be any of the known dehydrogenation catalysts, for example a catalyst containing one or more of the following metals: Li, Na, K, Cs, Mg, Al, In, Ga, Ag, Cu, Zn, Fe, Ni, Co, Mo, Ti, Pt or their oxides. Examples of specific dehydrogenation catalysts are: sodium or sodium compounds, aluminum oxide, alkali metal aluminate and/or alkaline earth metal aluminate, silver oxide, a catalyst comprising copper, zinc and sulphur, a catalyst comprising copper, zinc and selenium, a catalyst comprising zinc and/or indium, silver, and silver with copper and silicon.

Preference is given to dehydrogenation catalysts comprising silver because of their availability, low toxicity and general suitability. Suitable silver catalysts are known in the art. Suitable silver catalysts may comprise, for example, one or more of polycrystalline silver, Ag—SiO₂—Al₂O₃—ZnO (mass ratio of 20:55:2:8.3:16.5) and Ag—SiO₂—Al₂O₃—ZnO. Alternatively, the catalyst for converting at least a portion of the methanol to formaldehyde and hydrogen may comprise Cu—SiO₂ or Pt in Cu—SiO₂. Such catalysts may result in a high selectivity, high yield and/or high conversion rate.

The dehydrogenation catalyst may be employed in a membrane reactor.

Preferably, converting at least a portion of the methanol to formaldehyde and hydrogen is carried out under substantially anaerobic conditions. By “substantially anaerobic conditions”, it is meant that the reaction is performed without added oxygen such that substantially no water is produced during the conversion of methanol to formaldehyde, i.e. formaldehyde is produced substantially via the dehydrogenation route.

The substantial absence of an oxidant, in particular oxygen, may reduce the risk of operating in the methanol flammability envelope. Accordingly, the safety of the method may be improved.

Prior to recycling at least some of the recovered hydrogen to the feedstock gas stream, the recovered hydrogen is preferably purified. Such purification may remove species that may interfere unfavourably in the methanol conversion and/or poison catalysts used for the methanol conversion.

Preferably, recycling at least some of the recovered hydrogen to the feedstock gas stream comprises controlling the ratio of hydrogen to carbon oxides in the feedstock gas stream such that the stoichiometry number R is about 2, e.g. in the range 1.95 to 2.05. As noted above, such ratios may result in substantially complete conversion of the carbon oxides in the feedstock gas

The method preferably further comprises converting at least a portion of the recovered formaldehyde to one or more of urea formaldehyde resin, melamine resin, phenol formaldehyde resin, polyoxymethylene plastics, 1,4-butanediol, and methylene diphenyl diisocyanate. Since the method of the present invention is more energy efficient, environmentally friendly and/or lower cost in comparison to conventional formaldehyde productions methods, such species may be produced in a more energy efficient manner, in a more environmentally friendly manner and/or at lower cost than conventional methods to produce such species.

The method preferably further comprises converting at least a portion of the recovered formaldehyde to a formaldehyde-based resin for use as a textile finisher, preferably a textile finisher for providing crease-resistance to a textile.

The invention will now be described in relation to the following non-limiting examples.

### EXAMPLES

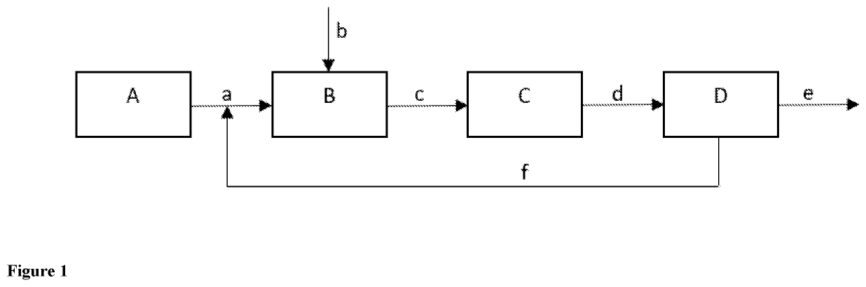

FIG. 1 shows a flow diagram of a plant suitable for carrying out the method of producing formaldehyde according to the present invention. At electrolysis unit A hydrogen a is generated by the electrolysis of water using renewable energy. The hydrogen a is then passed to the methanol synthesis unit B where it is combined with carbon dioxide b and converted to methanol c. The methanol is then passed to the distillation unit C where it is purified by distillation. The purified methanol d is then passed to the formaldehyde plant D where it is converted to formaldehyde e and hydrogen f. The formaldehyde e and hydrogen f are separately recovered, and the recovered hydrogen f is recycled back to the methanol synthesis unit B.

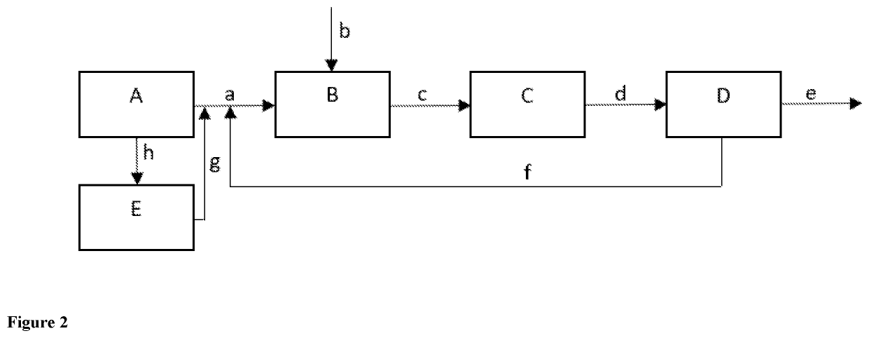

FIG. 2 shows a flow diagram of an alternative plant suitable for carrying out the method of producing formaldehyde according to the present invention. The plant is identical in structure to that shown in FIG. 1 except that the methanol synthesis unit B receives syngas g comprising hydrogen and carbon monoxide and optionally carbon dioxide. Syngas g is produced by the gasification of waste, biomass and/or coal in gasification unit E. Oxygen h produced by the electrolysis of water in the electrolysis unit A is passed to the gasification unit E for use in the gasification process. As will be appreciated, the passing of carbon dioxide b to the methanol synthesis unit B is now optional.

1. A method of producing formaldehyde, the method comprising:
generating electrolytic hydrogen from the electrolysis of water;
providing a feedstock gas stream comprising the electrolytic hydrogen and one or both of carbon monoxide and carbon dioxide;
converting at least a portion of the feedstock gas to methanol;
converting at least a portion of the methanol to formaldehyde and hydrogen;
separately recovering at least some of the formaldehyde and at least some of the hydrogen; and
recycling at least some of the recovered hydrogen to the feedstock gas stream.

2. The method of claim 1, wherein the electrolysis of water is carried out using renewable energy.

3. The method of claim 1, wherein the feedstock gas stream comprises electrolytic hydrogen and one or more of: carbon dioxide recovered from a waste stream, carbon dioxide recovered from a flue gas and carbon dioxide recovered from direct air capture.

4. The method of claim 1, wherein the feedstock gas stream comprises electrolytic hydrogen and a syngas comprising hydrogen, carbon monoxide and carbon dioxide.

5. The method of claim 4, wherein the syngas is formed by reforming of a hydrocarbon material with steam, or gasification of a carbonaceous material with oxygen and steam.

6. The method of claim 5, wherein the hydrocarbon material is natural gas or naphtha, and the carbonaceous material comprises one or more of biomass, municipal waste, plastics and coal.

7. The method of claim 4, wherein providing a feedstock gas comprises combining electrolytic hydrogen and the syngas, the hydrogen being generated from the electrolysis of water and the syngas being generated from the gasification of a carbonaceous material, wherein the gasification uses oxygen generated from the electrolysis of the water.

8. The method of claim 1, wherein converting at least a portion of the feedstock gas to methanol is carried out with a feedstock having a stoichiometry number R of about 2, wherein R is defined by the following formula: R=([H₂]−[CO₂])/([CO₂]+[CO]).

9. The method of claim 1, wherein the feedstock gas consists of electrolytic hydrogen and carbon dioxide.

10. The method of claim 1, wherein the feedstock gas comprises electrolytic hydrogen and both carbon monoxide and carbon dioxide.

11. The method of claim 1, wherein converting at least a portion of the hydrogen and at least a portion of the one or both of carbon monoxide and carbon dioxide to methanol comprises contacting the feedstock gas stream with a catalyst, wherein the contacting occurs at a temperature of from 200 to 330° C. and at a pressure of from 5 to 10 MPa.

12. The method of claim 11, wherein the catalyst comprises alumina-supported copper and zinc oxides.

13. The method of claim 1, wherein prior to converting at least a portion of the methanol to formaldehyde and hydrogen, the methanol is purified using distillation.

14. The method of claim 1, wherein converting at least a portion of the methanol to formaldehyde and hydrogen comprises contacting the methanol with a catalyst, wherein the contacting occurs at a temperature of from 300 to 800° C. and at a pressure of up to 5 MPa.

15. The method of claim 14, wherein the catalyst contains one or more of the following metals: Li, Na, K, Cs, Mg, Al, In, Ga, Ag, Cu, Zn, Fe, Ni, Co, Mo, Ti, Pt or their oxides.

16. The method of claim 14, wherein the catalyst comprises silver.

17. The method of claim 1, wherein converting at least a portion of the methanol to formaldehyde and hydrogen is carried out in the substantial absence of an oxidant.

18. The method of claim 1, wherein prior to recycling at least some of the recovered hydrogen to the feedstock gas stream, the recovered hydrogen is purified.

19. The method of claim 1, wherein recycling at least some of the recovered hydrogen to the feedstock gas stream comprises controlling the stoichiometry of the feedstock gas to have a stoichiometry number R of about 2, wherein R is defined by the following formula: R=([H₂]−[CO₂])/([CO₂]+[CO]).

20. The method of claim 1, further comprising converting at least a portion of the recovered formaldehyde to one or more of urea formaldehyde resin, melamine resin, phenol formaldehyde resin, polyoxymethylene plastics, 1,4-butanediol, and methylene diphenyl diisocyanate.
